(19) United States
(12) Patent Application Publication
(10) Pub. No.: US 2008/0188918 A1
(43) Pub. Date: Aug. 7, 2008

(54) METHOD AND SYSTEM FOR SUPPORTING IMPLANTATION OF BIVENTRICULAR CARDIAC PACEMAKERS

(76) Inventors: Jan Boese, Eckental (DE); Norbbert Rahn, Forchheim (DE)

Correspondence Address: SCHIFF HARDIN, LLP;PATENT DEPARTMENT, 6600 SEARS TOWER, CHICAGO, IL, 60606-6473, US

(21) Appl. No.: 11/671,549

(22) Filed: Feb. 6, 2007

(51) Int. Cl. A61N 1/05

(52) U.S. Cl. 607/119

(57) ABSTRACT

In a method and system for supporting implantation of the third electrode of a biventricular pacing system at an optimal location in the coronary sinus vessel tree, a 3D image of the left ventricle is acquired and a 3D image of the coronary sinus vessel tree is acquired, and these images are combined and displayed. A physician electronically interacts with the displayed image to indicate a marking thereon at a target position in the coronary sinus vessel tree for anchoring the third electrode. During implantation of the third electrode, real-time images of the coronary sinus vessel tree are obtained, on which the combined image with the marking therein is superimposed. The physician uses the real-time images with the marking superimposed thereon to guide implantation of the electrode, using an implantation tool, to the selected target position.

### BACKGROUND OF THE INVENTION

1. Field of the Invention

The present invention concerns a method and system for supporting implantation of cardiac pacemakers, and in particular for supporting implantation of biventricular cardiac pacemakers.

2. Description of the Prior Art

Tachycardia and bradycardia can be treated therapeutically with conventional pacemakers. Biventricular pacemaker therapy, also called cardiac resynchronization therapy (CRT), by contrast, is used to artificially create synchronization between the right and left ventricles that is not present, or is not always present, in patients having particular types of cardiac pathologies or insufficiencies. Biventricular pacing requires three electrodes. Two of these electrodes typically are placed in the right atrium and the right ventricle, respectively, at the locations where conventional cardiac pacemaker electrodes would normally be placed. The third electrode in biventricular pacing is positioned in a side branch of the coronary venous tree. For this purpose, a catheter is navigated through the coronary sinus into a communicating side branch, and the electrode is anchored in place. This electrode serves to stimulate the left ventricle (LV). For optimal stimulation of the left ventricle, it is important for the placement location of this third electrode to be correctly selected.

Conventionally, the placement of this third electrode requires a relatively time-consuming trial-and-error procedure, which can occupy many hours in certain individual cases.

A magnetically-controllable guide is commercially available from Stereotaxis that can be used to facilitate navigation in the coronary venous tree for supporting implantation of the third of the CRT electrodes. This known system, however, affords only limited possibilities for imaging in order to identify a suitable implantation location and catheter guidance to that location. In particular, the spatial correlation between the coronary venous system and the left ventricle is not known during the planning of this conventional procedure.

### SUMMARY OF THE INVENTION

It is an object of the present invention to provide a method and system for supporting implantation of biventricular cardiac pacemakers, in particular the third electrode of a CRT system that allow an optimal location for anchoring of this third electrode to be identified.

It is a further object of the present invention to provide such a method and system that supports catheter guidance of the third electrode to the aforementioned optimal location,

The above object is achieved in accordance with the present invention in a method and system wherein an image representation of the left ventricle is acquired, followed by an image representation of the coronary venous tree. The two image representations are acquired with the same coordinate system, either directly or in a manner that allows a translation between the respective coordinate systems of the image acquisitions. The two images are then combined, such as by image fusing, and the combined image is displayed. By interaction with the displayed image a physician identifies and marks a side branch of the coronary venous tree that appears to be optimal for stimulation of the left ventricle. The image of the coronary venous tree with this marking is then used for navigation of a catheter to place the third electrode of the CRT system at the marked location in the patient. For this purpose, the marked image can be superimposed with real-time fluoroscopic images of the patient obtained during the catheterization procedure. The third electrode is thereby guided to the marked location and is anchored at that location.

After anchoring a further image acquisition of the left ventricle can be acquired and the pumping action of the left ventricle can be determined therefrom. If the pumping action (function) appears satisfactory, no further steps are necessary. If the pumping action is sub-optimal, the aforementioned marking, guidance and anchoring steps can be repeated until an implantation location is determined that produces an optimal pumping action of the left ventricle.

### DESCRIPTION OF THE DRAWINGS

FIG. 1 is a flowchart showing the basic steps in the method in accordance with the present invention, implemented by the system in accordance with the present invention.

FIGS. 2A, 2B and 2C schematically illustrate images acquired in the planning portion of the inventive method.

FIG. 3A shows a target point marked in the combination image of FIG. 2C in accordance with the invention.

FIG. 3B schematically illustrates a displayed image in accordance with the present invention used to navigate a catheter or stylet wire to the marked location.

FIGS. 4A and 4B illustrate the use of the inventive method and system to avoid implantation at an area affected by scar tissue.

### DESCRIPTION OF THE PREFERRED EMBODIMENTS

The inventive method and system allow computed tomography (CT) images of a beating heart to be acquired during an interventional procedure for implanting the electrodes of a biventricular pacing system. The method and system can be directly integrated into existing angiography systems, such as the AXIOM Artis system that is commercially available from Siemens Medical Systems.

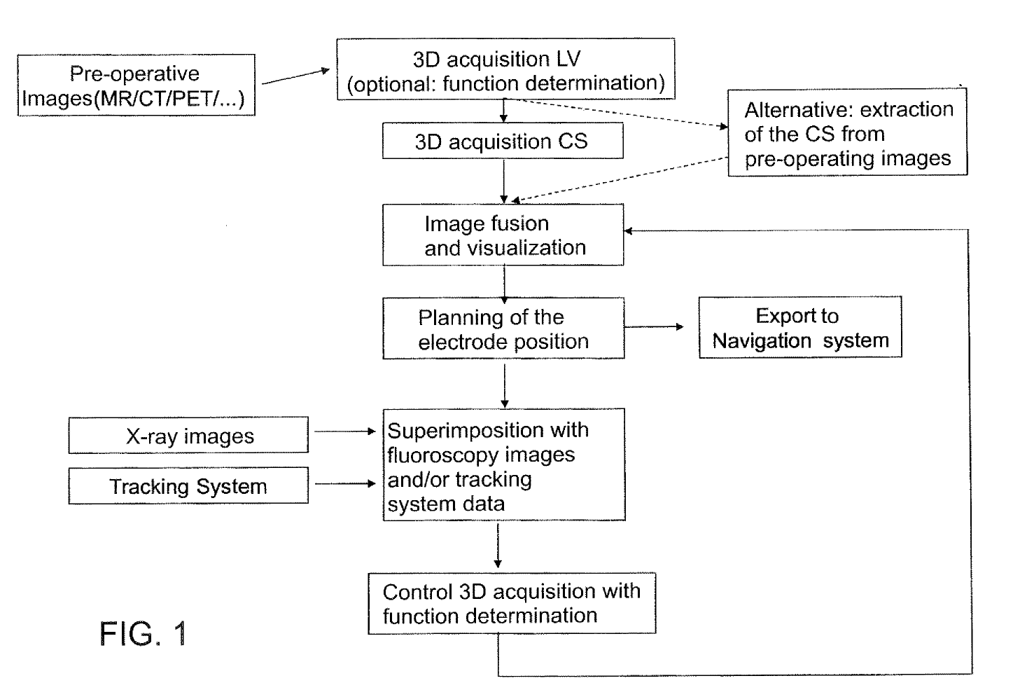

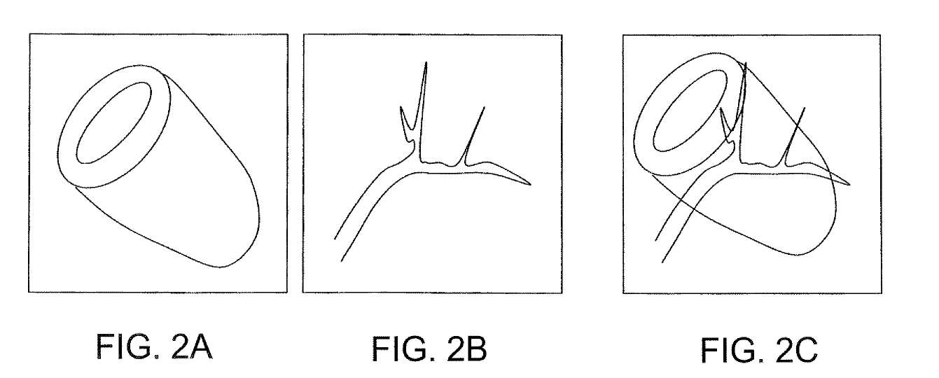

In step 1 shown in FIG. 1, a 3D dataset representing the left ventricle is acquired. This left ventricle is to be subsequently equipped with the three electrodes of a biventricular pacing system. For acquiring this 3D dataset, contrast agent can be injected either directly into the ventricle or intravenously. The resulting image is schematically illustrated in FIG. 2A.

Optionally, a determination of the functioning of the left ventricle can be made using the image of the left ventricle shown in FIG. 2A.

Alternatively pre-operative (pre-procedural) 3D image data of the left ventricle may exist before the 3D data acquisition in step 1. This is indicated as step 1A in FIG. 1. Such pre-operative 3D image data may be acquired, for example, by computed tomography (CT), magnetic resonance (MR), positron emission tomography (PET), or single photon emission computed tomography (SPECT). Such pre-operative images can be used to identify hypo-perfused tissue or necrotic scar tissue caused, for example, by a prior cardiac infarction. If such pre-operative images are acquired by CT or MR “late enhancement” image acquisition techniques can be used.

Optionally, a function determination of the left ventricle can be made from the 3D dataset acquired in step 1. If so, this 3D dataset can be a dynamic dataset so that heart movement with respect to time can be represented.

In step 2, a 3D image dataset of the coronary sinus (CS) is acquired, either using the same imaging modality and coordinate system as was used in step 1, or an imaging modality with a coordinate system with a known relationship (translation) relative to the coordinate system of the data in step 1. The 3D data acquisition of the coronary sinus can be accomplished, for example, by injecting contrast agent into the coronary sinus during the data acquisition and occluding backflow by means of an inflatable balloon.

Another option is to acquire a number of conventional 2D images of the coronary sinus, and to subject these 2D images (projections) to a 3D reconstruction, so that a 3D model of the coronary venous tree is created. For this purpose, image reconstruction techniques of the type described in U.S. Pat. No. 6,047,080 or PCT Application WO 2002/360113 can be used.

Another alternative, if the pre-operative images of step 1A are available, is to extract the image of the coronary sinus from the pre-operative images. This alternative is indicated as step 2A in FIG. 1.

The resulting image of the coronary sinus is schematically illustrated in FIG. 2B.

In step 3, the images obtained in steps 1 and 2, or their alternatives, are combined in the same coordinate system, and are displayed in this combined form. An image registration can be implemented before the combination to correct four patient movements. Known techniques for image fusion can be use to produce the combined image, such as weighted fading of the respective contributions from the two images. The resulting combination image is schematically illustrated in FIG. 2C.

Depending on whether the aforementioned alternatives are implemented, either of the datasets respectively acquired in steps 1 and 2 can be replaced by the images acquired in steps 1A and 2A. In such case, a 3D-registration procedure is implemented. This also facilities the identification of scar tissue that can be seen from the pre-operative images.

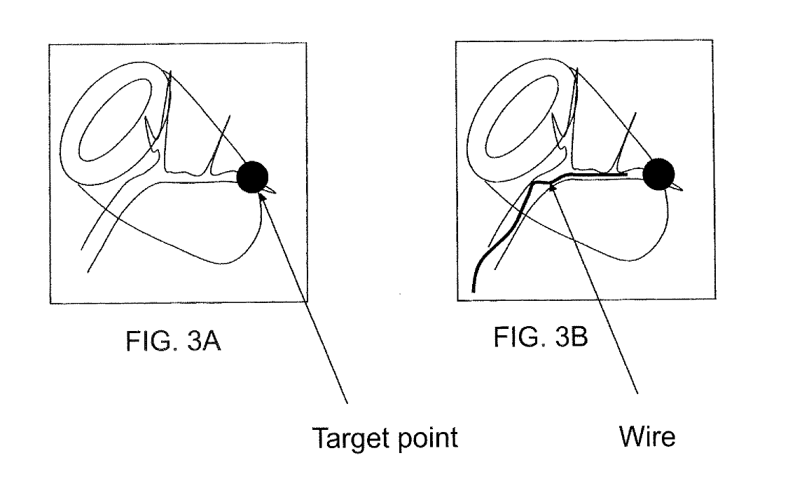

Using the combined representation shown in FIG. 2C, a physician then interacts with the displayed image, such as by using a touch screen or by cursor movement, to mark a side branch of the coronary venous tree that appears to be optimal for stimulation of the left ventricle. This represents a target point for anchoring of the third electrode of the biventricular pacing system. An image with an exemplary target point indicated therein is shown in FIG. 3A.

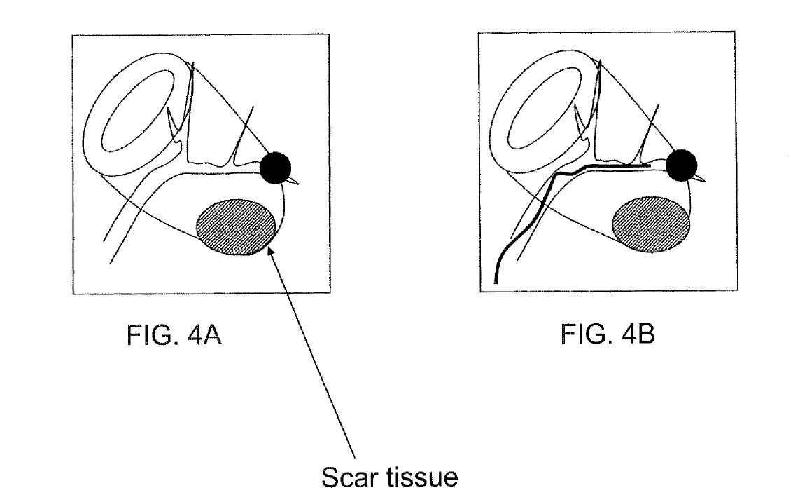

In the event that information (visualization) regarding scarred areas (for example, identified by scarring in the optional pre-operative 3D image) is available, as indicated in FIG. 4A, the target point can be identified and marked so that it will not be located in scarred tissue, since implantation at scarred tissue would put the success of the therapy at risk.

All of this ensues in the planning of the third electrode position, indicated as step 4 in FIG. 1. The image with the implantation (anchoring) position of the third electrode marked therein can then be used in the implantation procedure in step 5 of FIG. 1. As discussed below, this implantation procedure can be conducted with the use of a navigation system, in which case the image with the anchoring location marked therein is exported, in step 4A, to the navigation system.

In general, in step 5 the images of the coronary venous tree with the marking therein are used for navigation, with the goal being to be implant the third CRT electrode at the position identified and marked in step 4.

One alternative for implementing step 5 is to superimpose the data, including the marking, from step 4 on a real-time fluoroscopic image acquired from the patient during the implantation procedure. For this purpose, x-ray images (fluoroscopic images) are acquired in step 5A and are used in the implantation of step 5. A tracking system for the catheter or stylet wire can also acquire tracking system data in step 5B. The data from step 4 are superimposed with the images (data) used in the implantation procedure of step 5, in which the third CRT electrode is visualized (displayed) in real-time, using known 2D/3D fusion techniques.

Alternatively or additionally, intracardiac ultrasound can be used for the real-time representation of the third CRT electrode. For this purpose, intracardiac 2D ultrasound images are acquired and registered and superimposed with the 3D image data. The reconstruction of a 3D ultrasound image dataset from the intracardiac 2D ultrasound images is also possible, in which case the 3D ultrasound dataset can be registered and superimposed directly with the 3D image data from step 4.

A guide wire localization system can be used to localize the guide wire tip without x-ray radiation, as previously indicated using the tracking system of step 5B. This position (position data) is then mixed into the 3D image data.

Alternatively to such a tracking system, a navigation system can be used of the conventional type noted above, such a magnetic navigation system for controlling the guide wire. For this purpose, as noted above, the 3D data from step 4 are exported to the navigation system in step 4A.

An example of a display of the type that is presented in step 5 is shown in FIG. 3B. An alternative wherein the scar tissue is visualized is shown in FIG. 48.

As indicated in step 6, an image that represents functioning of the heart with the biventricular pacing system, including the third electrode thereof, anchored in their respective positions, can be acquired. Such images include at least two images respectively acquired at different points in time of the cardiac cycle, in order to assess the pumping function of the left ventricle with the third electrode at the position implanted in step 5. If the pumping function appears satisfactory, no further steps are necessary. If the pumping function is sub-optimal, a return to step 3 can be undertaken, and steps 4 and 5 can be repeated, as needed.

Compared to conventional techniques for implanting the electrodes, particularly the third electrode, of a CRT system, the system and method of the invention have several advantages. The inventive system and method allow an optimal selection of a suitable implantation location using the fused representation of the coronary veins and the left ventricle. The expenditure of time, the amount of administered contrast agent, and the x-ray dose to which the patient is exposed can all be reduced, thereby contributing to the overall success of the procedure. The therapy result can be iteratively improved due to the possibility of precise adjustments being made after a temporary anchoring.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for implanting an electrode in the coronary sinus tree of the heart of a patient, comprising the steps of:
obtaining a 3D image dataset representing a 3D image of the left ventricle of a heart;
obtaining a 3D dataset representing an image of the coronary sinus vessel tree of the heart;
electronically combining said 3D image of the left ventricle and said 3D image of the coronary sinus tree with the left ventricle and the coronary sinus tree in registration with each other, thereby obtaining a combined image;
visually displaying the combined image as a displayed image and allowing manual electronic interaction with the displayed image to mark, with a retained visual marking, a selected location in the coronary sinus vessel tree for anchoring said electrode;
obtaining and displaying real-time images of the heart of the patient and superimposing said real-time images at a display with said combined image with said marking; and
guiding an implantation tool, at least a portion of which is visible in said real time images, to bring said electrode to said target position using said real time images superimposed with said combined image with said marking

2. A method as claimed in claim 1 wherein the step of obtaining said 3D image dataset representing an image of the left ventricle comprises acquiring said 3D image dataset by administering a contrast agent that interacts with the left ventricle.

3. A method as claimed in claim 1 comprising implanting said electrode in an implantation procedure and wherein the step of obtaining said 3D image dataset representing an image of the left ventricle comprises acquiring a pre-operative image preceding said procedure.

4. A method as claimed in claim 3 comprising acquiring said pre-operative image using an imaging modality selected from the group consisting of computed tomography, magnetic resonance, positron emission tomography and single photon emission computed tomography.

5. A method as claimed in claim 3 wherein the step of obtaining said 3D image dataset representing an image of the coronary sinus vessel tree comprises electronically extracting said 3D image dataset representing said image of the coronary sinus vessel tree from said pre-operative image.

6. A method as claimed in claim 1 comprising combining said 3D image of the left ventricle with said 3D image of the coronary sinus vessel tree by image fusion.

7. A method as claimed in claim 1 comprising acquiring said 3D image dataset representing said image of the coronary sinus vessel tree by injecting contrast agent into the coronary sinus vessel tree and occluding backflow therefrom with an inflatable balloon.

8. A method as claimed in claim 1 comprising obtaining said 3D image dataset representing said image of said coronary sinus vessel tree by acquiring a plurality of 2D projections of the coronary sinus vessel tree and reconstructing said 3D dataset representing said 3D image of the coronary sinus vessel tree from said 2D projection images.

9. A method as claimed in claim 1 comprising identifying scarred cardiac tissue in said 3D image of the left ventricle and selecting said target position to avoid anchoring said electrode at said scarred tissue.

10. A method as claimed in claim 1 comprising acquiring said real-time images by x-ray fluoroscopy.

11. A method as claimed in claim 1 comprising generating a visual representation of said portion of said implantation tool using a tracking system.

12. A method as claimed in claim 1 comprising providing a navigation system with a dataset representing said combined image with said marking, and guiding said implantation tool using said navigation system and said real-time images.

13. A method as claimed in claim 1 comprising generating a visual representation of the left ventricle after anchoring said electrode in said coronary sinus vessel tree and determining functioning of said left ventricle therefrom and, if necessary, repeating at least the steps of selecting said target position and guiding said implantation tool to anchor said electrode at said target position based on the determined functioning of the left ventricle.

14. A system for implanting an electrode in the coronary sinus tree of a heart of a patient, comprising:
a unit that obtains a 3D image dataset representing a 3D image of the left ventricle of a heart and that obtains a 3D dataset representing an image of the coronary sinus vessel tree of the heart;
a computer that electronically combines said 3D image of the left ventricle and said 3D image of the coronary sinus tree with the left ventricle and the coronary sinus tree in registration with each other, thereby obtaining a combined image;
a display connected to said computer that visually displays the combined image as a displayed image and allows manual electronic interaction with the displayed image to mark, with a retained visual marking, a selected location in the coronary sinus vessel tree for anchoring said electrode;
an imagining unit that obtains and displays real-time images of the heart of the patient and superimposing said real-time images at a display with said combined image with said marking; and
said display allowing guiding of an implantation tool, at least a portion of which is visible in said real time images, to bring said electrode to said target position using said real time images superimposed with said combined image with said marking

15. A system as claimed in claim 14 wherein said unit that obtains said 3D image dataset representing an image of the left ventricle is an imaging unit that acquires said 3D image dataset with administration of a contrast agent that interacts with the left ventricle.

16. A system as claimed in claim 14 wherein said electrode is implanted in an implantation procedure and wherein said unit that obtains said 3D image dataset representing an image of the left ventricle is an imaging unit that acquires a pre-operative image of the left ventricle preceding said procedure.

17. A system as claimed in claim 16 wherein said imaging unit that acquires said pre-operative image using an imaging modality selected from the group consisting of computed tomography, magnetic resonance, positron emission tomography and single photon emission computed tomography.

18. A system as claimed in claim 16 wherein said unit that obtains said 3D image dataset representing an image of the coronary sinus vessel tree is a computer that electronically extracts said 3D image dataset representing said image of the coronary sinus vessel tree from said pre-operative image.

19. A system as claimed in claim 14 wherein said computer combines said 3D image of the left ventricle with said 3D image of the coronary sinus vessel tree by image fusion.

20. A system as claimed in claim 14 comprising wherein said unit that obtains said 3D image dataset representing said image of the coronary sinus vessel tree is an imaging unit with a contrast agent injector agent that injects contrast agent into the coronary sinus vessel tree and occludes backflow therefrom with an inflatable balloon.

21. A system as claimed in claim 14 wherein said unit that obtains said 3D image dataset representing said image of said coronary sinus vessel tree is an imaging unit that acquires a plurality of 2D projections of the coronary sinus vessel tree and a computer that reconstructs said 3D dataset representing said 3D image of the coronary sinus vessel tree from said 2D projection images.

22. A system as claimed in claim 14 wherein said display allows identification of scarred cardiac tissue in said 3D image of the left ventricle and selection of said target position to avoid anchoring said electrode at said scarred tissue.

23. A system as claimed in claim 14 wherein said imaging unit that acquires said real-time images is an x-ray fluoroscopy system.

24. A system as claimed in claim 14 comprising a tracking system that generates a visual representation of said portion of said implantation tool at said display.

25. A system as claimed in claim 14 comprising a navigation system provided with a dataset representing said combined image with said marking, that allows guiding of said implantation tool using said navigation system and said real-time images.

26. A system as claimed in claim 14 comprising an imaging unit that generates a visual representation of the left ventricle after anchoring said electrode in said coronary sinus vessel tree allowing a determination of functioning of said left ventricle therefrom for, if necessary, repeating at least selection of said target position and guidance of said implantation tool to anchor said electrode at said target position based on the determined functioning of the left ventricle.
